SEQUENCE LISTING (2 sequences)

SEQ ID NO: 1 - protein, 449 aa, synthetic construct
QVTLKESGPTLVKPTQTLTLTCSFSGFSLSTSGMGIGWIRQPPGKALEWLAHIWWDDDKRYNPALKSRLTISKDTSKNQVVLTMTNMDPVDTATYYCARHYGYDPYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 215 aa, synthetic construct
EIVLTQSPGTLSLSPGERATLSCTASSSVSSSYLHWYQQKPGKAPKLLIYRTSNLASGVPSRFSGSGSGTDYTLTISSLQPEDFATYYCHQYHRSPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0051438 A1
(43) Pub. Date: Feb. 13, 2025

(54) METHOD FOR USING INFLAMMATORY MARKERS TO GUIDE ANTI-CLEVER-1 BASED CANCER TREATMENT

(71) Applicant: Faron Pharmaceuticals Oy, Turku (FI)

(72) Inventors: Juho JALKANEN, Mietoinen (FI); Teppo HUTTUNEN, Turku (FI)

(73) Assignee: Faron Pharmaceuticals Oy, Turku (FI)

(21) Appl. No.: 18/717,841

(22) PCT Filed: Dec. 5, 2022
(86) PCT No.: PCT/FI2022/050809

§ 371 Date: Jun. 7, 2024

(30) Foreign Application Priority Data
Dec. 7, 2021 (FI) 20216250

(51) Int. Cl. C07K 16/28; G01N 33/574; G01N 33/68

(52) U.S. Cl. CPC C07K 16/28; G01N 33/57488; G01N 33/6863

(57) ABSTRACT

A method for choosing a patient to anti-CLEVER-1 therapy prior to beginning anti-CLEVER-1 therapy using one or several of the inflammatory markers selected from interferon gamma (IFNγ), tumour necrosis factor alpha (TNF-α), interleukin 6 (IL-6), interleukin 8 (IL-8) and C-reactive protein. When the measured level of the inflammatory marker is substantially within the reference values of the inflammatory marker or lower than the average reference value of the inflammatory marker, it is an indication to choose the patient and begin the anti-CLEVER-1 therapy.

### FIELD OF THE INVENTION

The present invention relates to use of one or more inflammatory markers to guide anti-CLEVER-1 based cancer treatment and a method for choosing a patient to anti-CLEVER-1 therapy prior to of anti-CLEVER-1 therapy.

### BACKGROUND OF THE INVENTION

Anti-PD-1, anti-PD-L1 and anti-CTLA-4 immune-checkpoint inhibitors are extensively used in clinical patient care. Currently, immune checkpoint inhibitors targeting CTLA-4 and the PD-1/PD-L1 axis are approved for clinical use, and while highly efficacious in about 10-20% of patients with melanoma and certain other tumors, several other important cancer types (such as prostate, breast and colorectal cancer) remain refractory to them, and there is no clear biomarker available that could differentiate responders from non-responders and guide treatment [1]. Patients responding favorably to checkpoint inhibition usually have a pre-existing antitumor immune response, which is characterized by high density of interferon gamma (IFNg) producing CD8⁺ T cells, expression of PD-L1 in tumor-infiltrating immune cells, and high mutational load. Tumors that do not respond to immune checkpoint blockage show either a stromal T cell phenotype where infiltration of T cells (TIL) into tumors or activation of T cells in the tumor microenvironment (TME) is prevented by immunosuppressive stromal compartments, or a non-inflamed phenotype characterized by low T cell infiltration, low mutational load and high proliferation of tumor cells. The tumors can be classified immunologically (inflamed vs. non-inflamed) based on the presence of tumor infiltrating cytotoxic CD8 T cells [2]. The inflamed tumors show high mutational load, high IFNg and PD-L1 expression, and respond favorably to immune checkpoint blocking therapies. IFNg produced by T cells is considered necessary for immunological checkpoint inhibitors to work as anti-cancer agents.

Innate immune cells such as macrophages, however, can dampen T cell activation and contribute to tumor progression despite high mutational load. The macrophages that contribute to tumor-related immunosuppression and provide tumor growth supporting signals may be highly eligible candidates for targeted therapies, since these cells are abundantly present in various tumors, they are very plastic and can be converted into pro-inflammatory macrophages supporting T cell activation and tumor rejection [3, 4]. To date, macrophage targeted strategies under clinical development utilize macrophage colony-stimulating factor receptor inhibition to deplete macrophage populations in tumors [5]. However, resistances to these approaches have already been reported [6]. Thus, there is a need to find novel ways to utilize these cells to induce tumor cell killing by the immune system.

In recent years, increasing attention has been paid to the contribution of scavenger receptors in regulating macrophage responses to different stimuli. CLEVER-1 (also known as STABILIN-1) is a multifunctional molecule conferring scavenging ability on a subset of anti-inflammatory macrophages [7, 8]. In these cells, it is involved in receptor-mediated endocytosis and recycling, intracellular sorting, and transcytosis of altered and normal self-components. More recently, it has been found that the growth and metastases are attenuated in Stab1^{-/-} (Clever-1 knock out) mice in several tumor models, and in mice treated with anti-CLEVER-1 therapy [9, 10]. In addition, combination treatments with an anti-CLEVER-1 agent together with an anti-PD-1 agent has shown to produce anti-tumor responses in mouse models of triple negative breast cancer and colorectal cancer [10]. The first anti-CLEVER-1 antibody named bexmarilimab has begun clinical development showing promising anti-tumor responses in its first-in-human trial [11].

### SUMMARY OF THE INVENTION

Now, it has been surprisingly found out that opposite to classical checkpoint inhibitors anti-PD-1, anti-PD-L1 and anti-CTLA-4, anti-CLEVER-1 therapy is effective in non-inflamed tumour types characterized by low interferon gamma (IFNg) levels as well as low levels of other inflammatory cytokines. It has been recently found that approx. 30% of patients with advanced melanoma, gastric cancer and cholangiocarcinoma response favorably to anti-CLEVER-1 IgG4 antibody bexmarilimab (FP-1305) in a Phase I/II study (clinicaltrials.gov NCT03733990: A Study to Evaluate Safety, Tolerability and Preliminary Efficacy of FP-1305 in Cancer Patients (MATINS)). Now, it has been further observed that these patients, which respond to the anti-CLEVER-1 antibody therapy in MATINS trial can be efficiently identified by low IFNg values compared to high values at baseline, this is totally opposite to what has so far been seen in the field of immuno-oncology. Similarly, it has been observed that tumour necrosis factor alpha (TNF-α) levels in serum sample are low at baseline or substantially same as the reference value in patients who experienced clinical benefit in the MATINS trial. In addition, based on a receiver operating characteristic (ROC) curve analysis, 83% of patients who have low levels of IFNg and TNF-α are expected to experience clinical benefit when treated with anti-CLEVER-1 antibody bexmarilimab. Further, the likelihood of predicting response to anti-CLEVER-1 antibody bexmarilimab increases to 91%, if classical pro-inflammatory cytokines such as interleukin 6 (IL-6) and/or interleukin 8 (IL-8) are added to the ROC analyses. Also, it has been observed that a standard inflammatory laboratory value C-reactive protein (CRP) has excellent ability to reflect a non-inflammatory cancer type, that is responsive to anti-CLEVER-1 antibody bexmarilimab.

Hence, the present invention provides a novel inflammatory marker-based treatment for cancer patients and thus reduce or even eliminate the abovementioned problems in defining non-responders from responding patients. The present invention gives the possibility of probing the molecular landscape of solid tumors via a simple blood draw.

The present invention is based on measuring one or more inflammatory markers prior to beginning anti-CLEVER-1 therapy, to choose the patient that should receive anti-CLEVER-1 therapy, and especially to choose the patient that will benefit from receiving anti-CLEVER-1 therapy comprising an administration of an agent capable of binding to CLEVER-1, preferably anti-CLEVER-1 antibody, more preferably anti-CLEVER-1 antibody bexmarilimab. The present invention provides a tool to choose who should receive anti-CLEVER-1 treatment, preferably anti-CLEVER-1 antibody treatment, more preferably anti-CLEVER-1 antibody bexmarilimab treatment. The present invention provides use of one or more inflammatory markers selected from the group consisting of interferon gamma (IFNg), tumour necrosis factor alpha (TNF-α), interleukin 6 (IL-6), interleukin 8 (IL-8) and C-reactive protein (CRP) to guide anti-CLEVER-1 based cancer treatment.

A typical method according to the present invention for choosing a patient to anti-CLEVER-1 therapy prior to beginning anti-CLEVER-1 therapy, which anti-CLEVER-1 therapy comprises an administration of an agent capable of binding to CLEVER-1, preferably anti-CLEVER-1 antibody, more preferably anti-CLEVER-1 antibody bexmarilimab in a patient, the method comprises at least the following steps of
providing a blood sample obtained from the patient at a first point in time prior to beginning the anti-CLEVER-1 therapy,
measuring a level of one or more inflammatory marker selected from the group consisting of interferon gamma (IFNg), tumour necrosis factor alpha (TNF-α), interleukin 6 (IL-6), interleukin 8 (IL-8) and C-reactive protein (CRP) from the obtained sample, and
comparing the measured level of the inflammatory marker to a reference value of said inflammatory marker,
   wherein the measured level of the said inflammatory marker, which is substantially within the reference values of the inflammatory marker or lower than the average reference value of the inflammatory marker, is an indication to choose the patient and begin the anti-CLEVER-1 therapy.

A method according to the present invention can include measuring of only one inflammatory marker selected from the group consisting of IFNg, TNF-α, IL-6, IL-8 and CRP, but it may also be a combination of two or more these inflammatory markers. For making the decision to begin the anti-CLEVER-1 therapy, these inflammatory markers need to be at the baseline or below the baseline or substantially same as the reference value or lower than the reference value of the said inflammatory marker. If the level of the inflammatory marker is abnormally high value compared to the baseline, it may lead to non-responsiveness to anti-CLEVER-1 therapy, e.g. anti-CLEVER-1 antibody therapy, such as anti-CLEVER-1 antibody bexmarilimab therapy.

Further, the present invention provides a method for treating cancer patient, which method comprises at least the following steps of
obtaining a blood sample from the cancer patient at a first point in time prior to beginning the anti-CLEVER-1 therapy,
measuring a level of one or more inflammatory marker selected from the group consisting of interferon gamma (IFNg), tumour necrosis factor alpha (TNF-α), interleukin 6 (IL-6), interleukin 8 (IL-8) and C-reactive protein (CRP) from the obtained sample,
comparing the measured level of the inflammatory marker to a reference value of said inflammatory marker, and
beginning an administration of an agent capable of binding to CLEVER-1, preferably an anti-CLEVER-1 antibody, more preferably anti-CLEVER-1 antibody bexmarilimab in a patient, when the measured level of the said inflammatory marker is substantially within the reference values of the inflammatory marker or lower than the average reference value of the inflammatory marker.

Based on the present invention, an agent capable of binding CLEVER-1, preferably anti-CLEVER-1 antibody, and more preferably a humanized monoclonal immunoglobulin G4κ antibody bexmarilimab can be efficiently used in the treatment of cancer, especially in the treatment of advanced solid tumours, in a cancer patient. In one aspect of the present invention anti-CLEVER-1 antibody, preferably a humanized monoclonal immunoglobulin G4κ antibody bexmarilimab is used for the treatment of cancer, preferably for treatment of advanced solid tumours in a patient which shows the reference value within the reference values of the inflammatory marker or lower than the average reference value of the inflammatory marker prior to the beginning of an anti-CLEVER-1 therapy, which inflammatory marker comprise interferon gamma (IFNg), tumour necrosis factor alpha (TNF-α), interleukin 6 (IL-6), interleukin 8 (IL-8) and/or C-reactive protein (CRP).

### BRIEF DESCRIPTION OF THE DRAWINGS

FIGS. 1-3. The inflammatory marker data extracted from expansion cohorts of the first-in-human bexmarilimab trial (MATINS) indicates that subjects that showed clinical benefit from bexmarilimab in the Kaplan-Meier survival analysis (survival probability 0.8 in FIG. 1) have clearly lower baseline serum IFNg (FIG. 2) and TNF-α levels (FIG. 3). In FIGS. 2 and 3, right side diagrams present subjects benefit from bexmarilimab in the Kaplan-Meier survival analysis (Disease control rate (DCR)=yes).

FIG. 4. ROC analysis comparing pre-treatment IFNg and TNF-α levels in patients that had a survival benefit from bexmarilimab to those that did not in the first-in-human bexmarilimab trial (MATINS). Low IFNg and TNF-α values associate with clinical benefit. The predictive value of these biomarkers was excellent (AUC 0.83, p<0.0001).

FIG. 5. ROC analysis comparing pre-treatment IFNg, TNFa, IL-6 and IL-8 levels in patients that had a survival benefit from bexmarilimab to those that did not in the first-in-human bexmarilimab trial (MATINS). Low values associate with clinical benefit. The predictive value of these biomarkers was excellent (AUC 0.91, p<0.0001).

FIG. 6. ROC analysis comparing pre-treatment CRP level in patients that had a survival benefit from bexmarilimab to those that did not in the first-in-human bexmarilimab trial (MATINS). Low CRP values associate with clinical benefit. The predictive value of CRP alone is relatively good (AUC 0.78, p=0.036).

FIG. 7. The amino acid sequences of light and heavy chains of anti-CLEVER-1 antibody bexmarilimab.

### DETAILED DESCRIPTION OF THE INVENTION

CLEVER-1 is a protein disclosed in the patent publication WO 03/057130, Common Lymphatic Endothelial and Vascular Endothelial Receptor-1. It is a binding protein that mediates adhesion of lymphocytes to endothelium in both the systemic vasculature and in the lymphatics. By blocking the interaction of CLEVER-1 and its lymphocyte substrate, it is possible to simultaneously control lymphocyte recirculation and lymphocyte migration, and related conditions such as inflammation, at the site of lymphocyte influx into, and efflux from, the tissues.

The term “an agent capable of binding to CLEVER-1” refers to agents including antibodies and fragments thereof, peptides or the like, which are capable of binding to CLEVER-1 for blocking the interaction of CLEVER-1 and malignant tumor cells. The agent may also be any other inhibitor, such as small molecule inhibitor or macromolecule having an adequate affinity to bind to CLEVER-1 receptor and to inhibit the protein activity. The term “an antibody or a fragment thereof” is used in the broadest sense to cover an antibody or a fragment thereof which are capable to bind CLEVER-1 molecule in an individual. Especially, it shall be understood to include chimeric, humanized or primatized antibodies, as well as antibody fragments and single chain antibodies (e.g. Fab, Fv), so long they exhibit the desired biological activities. Particular useful agents are anti-CLEVER-1 antibodies and fragments thereof. Therefore, according to an embodiment of the present invention an agent capable of binding to CLEVER-1, i.e. anti-CLEVER-1 agent, is selected from the group consisting of an antibody or a fragment thereof, peptide(s), macromolecule and any combination thereof. According to the present invention “anti-CLEVER-1 treatment” or “anti-CLEVER-1 therapy” refers to the treatment comprising administration of at least one agent capable of binding CLEVER-1, preferably anti-CLEVER-1 antibody. In a preferred embodiment of the present invention, anti-CLEVER-1 therapy refers to anti-CLEVER-1 antibody therapy.

According to an embodiment of the invention, an anti-CLEVER-1 antibody is a therapeutic humanized anti-CLEVER-1 antibody. According to an embodiment of the present invention an anti-CLEVER-1 antibody is a humanized monoclonal CLEVER-1 antibody, previously presented in the patent publication WO2017/182705.

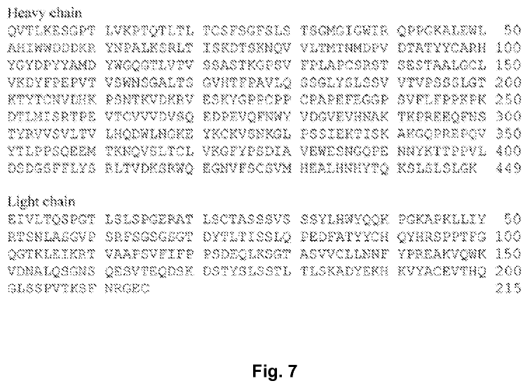

In an embodiment of the present invention, an anti-CLEVER-1 antibody is a humanized monoclonal immunoglobulin G4κ antibody bexmarilimab (International Nonproprietary Name (INN)) as disclosed in WHO Drug Information, Vol. 33, No. 4, pages 814-815 (2019)), or bexmarilimab variant or the antibody in a bexmarilimab biosimilar. As used herein, “bexmarilimab” means the humanized monoclonal IgG4 antibody with the structure described in WHO Drug Information, Vol. 33, No. 4, pages 814-815 (2019). A humanized IgG4 monoclonal anti-CLEVER-1 antibody bexmarilimab comprises an amino acid sequence SEQ ID NO:1 of a heavy chain and an amino acid sequence SEQ ID NO:2 of a light chain. The amino acid sequences of heavy chain (SEQ ID NO:1) and light chain (SEQ ID NO:2) of anti-CLEVER-1 antibody bexmarilimab are also shown in FIG. 7.

A bexmarilimab biosimilar means a biological product which is approved by a regulatory agency in any country for marketing as a bexmarilimab biosimilar. In an embodiment, a bexmarilimab biosimilar comprises a bexmarilimab variant as the drug substance. In an embodiment, a bexmarilimab biosimilar has substantially the same amino acid sequence of heavy and light chains as bexmarilimab. As used herein, a “bexmarilimab variant” means an antibody which comprises sequences of heavy chain and light chain that are identical to those in bexmarilimab, except for having one or more conservative amino acid substitutions at positions that are located outside of the light chain CDRs and/or one or more conservative amino acid substitutions that are located outside of the heavy chain CDRs, e.g. the variant positions are located in the framework regions or the constant region. In other words, bexmarilimab and a bexmarilimab variant comprise identical CDR sequences, but differ from each other due to having a conservative amino acid substitution at other positions in their full-length light and heavy chain sequences. A bexmarilimab variant is substantially the same as bexmarilimab with respect to binding affinity to CLEVER-1.

According to an embodiment of the present invention, a cell line producing the therapeutic anti-CLEVER-1 antibody bexmarilimab (FP-1305) has been deposited on 27 May 2020 under the terms of the Budapest Treaty on the International Recognition of the Deposit of Micro-organisms for the Purposes of Patent Procedure with the DSMZ-German Collection of Microorganisms and Cell Cultures GmbH, Inhoffenstrasse 7B, D-38124 Braunschweig, Germany, and has the accession number DSM ACC3361. The present invention is not to be limited in scope by the culture deposited, since the deposited embodiment is intended as a single illustration of one aspect of the invention and any culture that is functionally equivalent is within the scope of this invention. The deposit of material herein does not constitute an admission that the written description herein contained is inadequate to enable the practice of any aspect of the invention, including the best mode thereof, nor is it to be construed as limiting the scope of the claims to the specific illustration that it represents.

According to an embodiment of the invention, a sample obtained at a first point in time prior to the administration of an agent capable of binding to CLEVER-1, preferably prior to the administration of anti-CLEVER-1 antibody, such as anti-CLEVER-1 antibody bexmarilimab to a patient refers to a sample obtained prior to the first administration of an agent capable of binding to CLEVER-1, preferably prior to the first administration of anti-CLEVER-1 antibody, such as anti-CLEVER-1 antibody bexmarilimab. A sample obtained from the patient refers to a blood sample.

According to an embodiment of the present invention, a method comprises measuring one or more inflammatory markers selected from the group consisting of interferon gamma (IFNg), tumour necrosis factor alpha (TNF-α), interleukin-6 (IL-6), interleukin-8 (IL-8) or C-reactive protein (CRP) from the obtained sample and using the measured value of one inflammatory marker or a combination of two or more the measured values of the inflammatory markers to choose to treat a patient with an anti-CLEVER-1 treatment, preferably with anti-CLEVER-1 antibody treatment. The level of the inflammatory markers can be measured from serum and/or plasma. For making decision to begin anti-CLEVER-1 therapy, preferably anti-CLEVER-1 antibody therapy, the level of these inflammatory markers need to be at average range of the reference or lower than the average reference value of the inflammatory marker. Based on the findings of the present invention abnormally high values compared to the reference value of the said inflammatory marker may lead to non-responsiveness to an agent capable of binding to CLEVER-1, e.g. to anti-CLEVER-1 antibody bexmarilimab. Based on the present invention, only one inflammatory marker can be used in the decision making for beginning of anti-CLEVER-1 therapy, preferably anti-CLEVER-1 antibody therapy, but the reliability of the possible response to the therapy improves if using the combination of two or more inflammatory markers.

According to an embodiment of the present invention the method comprises measuring at least the level of interferon gamma (IFNg) and tumour necrosis factor alpha (TNF-α). In an embodiment of the present invention, the method comprises measuring the level of interferon gamma (IFNg) and/or tumour necrosis factor alpha (TNF-α) and also the level of interleukin 6 (IL-6) and/or interleukin 8 (IL-8). In an embodiment of the present invention, the method comprises measuring the level of interferon gamma (IFNg), tumour necrosis factor alpha (TNF-α), interleukin 6 (IL-6) and/or interleukin 8 (IL-8) in combination with measuring of the level of C-reactive protein (CRP). According to an embodiment of the present invention, the level of C-reactive protein (CRP) alone may be used to evaluation necessity to begin anti-CLEVER-1 therapy, preferably anti-CLEVER-1 antibody therapy.

According to the present invention, measurements of the inflammatory markers can be carried out by conventional methods known in the art. There are available a variety of the analyzing methods and devices for the purposes. The reference value or baseline value of the inflammatory marker is dependent on the measurement method. According to the present invention reference value or baseline value of the inflammatory marker means a normal value or a value within the certain range, which has been defined to be normal or average conventional value of said inflammatory marker in the field.

According to an embodiment of the invention, anti-CLEVER-1 therapy comprises an administration of therapeutically effective amounts of an agent capable of binding to Clever-1, preferably an anti-CLEVER-1 antibody, more preferably anti-CLEVER-1 antibody bexmarilimab. The term “therapy”, “treatment” or “treating” shall be understood to include complete curing of a disease or disorder, as well as amelioration or alleviation of said disease or disorder. In an embodiment according to the present invention, anti-CLEVER-1 therapy refers to anti-CLEVER-1 antibody therapy, which comprises an administration of therapeutically effective amounts of an anti-CLEVER-1 antibody, preferably anti-CLEVER-1 antibody bexmarilimab. The term “therapeutically effective amount” is meant to include any amount of an agent according to the present invention that is sufficient to bring about a desired therapeutic result. “Administering” refers to the physical introduction of a composition comprising said therapeutic agents to an individual, using any of the various methods and delivery systems known to those skilled in the art. The agents to be used in the present invention may be administered by any means that achieve their intended purpose. For example, administration may be intravenous, intramuscular, intraperitoneal, intratumoral, subcutaneous or other parenteral routes of administration, for example by injection. In addition to the pharmacologically active compounds, the pharmaceutical preparations of said agents preferably contain suitable pharmaceutically acceptable carriers comprising excipients and auxiliaries that facilitate processing of the active agents into preparations that can be used pharmaceutically. The dose chosen should be sufficient to reduce or inhibit malignant tumor growth and/or inhibit metastasis formation.

A method according to the present invention for making decision to treat a cancer patient prior to beginning anti-CLEVER-1 therapy and also a method of treating cancer is applicable to all forms of cancers. Thus, any malignant tumor or metastasis can be treated. In an embodiment according to the present invention, anti-CLEVER-1 therapy is used for treating advanced solid tumours. According to an embodiment of the present invention, anti-CLEVER-1 therapy is used at least for treating cancer selected from melanoma, gastric cancer and cholangiocarcinoma.

### EXPERIMENTAL PART

### Human Studies on CLEVER-1 Inhibition for the Treatment of Cancer

CLEVER-1 inhibiting agent, an anti-CLEVER-1 antibody FP-1305 is currently being tested for safety and preliminary efficacy in a Phase I/II study in patient with advanced solid tumors (clinicaltrials.gov NCT03733990: A Study to Evaluate Safety, Tolerability and Preliminary Efficacy of FP-1305 in Cancer Patients (MATINS)).

An anti-CLEVER-1 antibody FP-1305 is a humanized monoclonal CLEVER-1 antibody, previously presented in the patent publication WO2017/182705. More precisely, FP-1305 (DSM ACC3361) is a humanized monoclonal immunoglobulin G4κ antibody bexmarilimab (International Nonproprietary Name (INN)) as disclosed in WHO Drug Information, Vol. 33, No. 4 (2019) as proposed INN and in WHO Drug Information, Vol. 34, No. 3 (2020), pages 699-700 as recommended INN).

In the present study, first (pre-dose) serum sample has been taken from the patients prior to initiating the administration of FP-1305 (bexmarilimab). IFNg, TNF-α, IL-6 and IL-8 levels and C-reactive protein (CRP) are measured from a standard serum sample using conventional measurements methods known in the art.

In the present study, the patients receive anti-CLEVER-1 antibody at a dose of 1 mg/kg given every three weeks for an un-limited time until disease progression.

### Results

In the MATINS study, approx. 30% of patients with advanced melanoma, gastric cancer and cholangiocarcinoma responded favorably to anti-CLEVER-1 IgG4 antibody bexmarilimab (FP-1305).

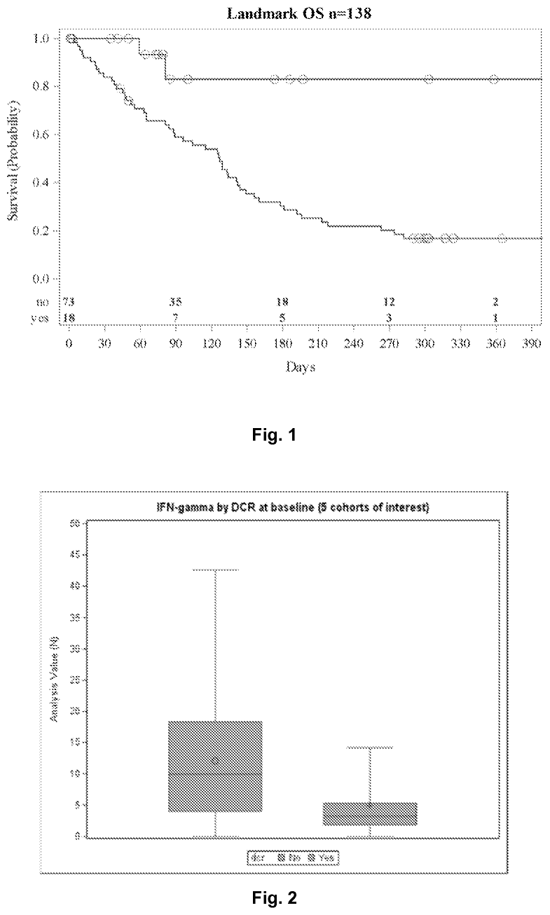

As shown in FIGS. 1-3, the biomarker data extracted from these expansion cohorts of the first-in-human bexmarilimab trial (MATINS) indicates that subjects that showed clinical benefit from bexmarilimab in the Kaplan-Meier survival analysis have clearly lower baseline serum IFNg and TNF-α levels. This indicates that bexmarilimab could be especially effective in so-called highly immunosuppressed non-inflammatory “cold” tumours.

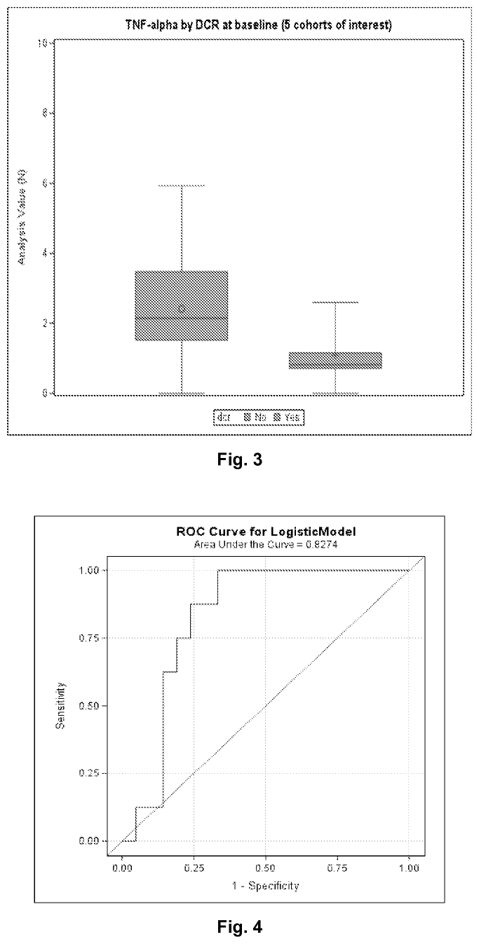

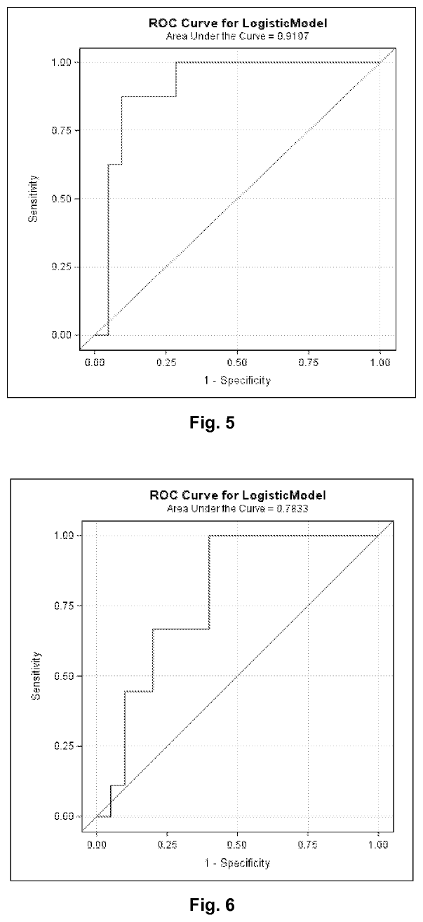

More detailed, among the 30 patients, all of whom were heavily pre-treated and refractory to checkpoint inhibitors, there were nine who experienced clinical benefit (partial response or stable disease) and 21 who did not experience clinical benefit following treatment with bexmarilimab. At baseline, the nine patients who experienced clinical benefit had average serum interferon gamma (IFNg) levels of 5.11 pg/ml (SD, +/−4.99 pg/ml) compared to 13.07 pg/ml (SD, +/−13.26) for patients that did not experience clinical benefit following treatment with bexmarilimab (FIG. 2). Similarly, serum tumour necrosis factor alpha (TNF-α) levels at baseline in patients who experienced clinical benefit were 0.96 pg/ml (SD, +/−0.74 pg/ml) compared to 2.37 pg/ml (SD, +/−1.43 pg/ml) in patients that did not experience clinical benefit following treatment with bexmarilimab (FIG. 3). Based on a receiver operating characteristic (ROC) curve analysis, 83% of patients who have low levels of IFNg and TNF-α are expected to experience clinical benefit when treated with bexmarilimab (FIG. 4). The likelihood of predicting response to bexmarilimab increases to 91% if classical pro-inflammatory cytokines such as interleukin 6 (IL-6) and/or interleukin 8 (IL-8) are added to the ROC analyses (FIG. 5). Also, a standard inflammatory laboratory value C-reactive protein (CRP) has excellent ability to reflect a non-inflammatory cancer type, that is responsive to bexmarilimab (FIG. 6).

Hence, based on the above analysis of the patients that showed clinical benefit from anti-CLEVER-1 antibody bexmarilimab in the Kaplan-Meier survival analysis, it can be concluded that the decision to begin the anti-CLEVER-1 antibody therapy may be made using the said inflammatory markers. The response to anti-CLEVER-1 antibody therapy will be better if the level of the said inflammatory markers is at the normal average levels or below the reference values prior to the beginning of the therapy. If the levels of the inflammatory markers are abnormally high values compared to the reference values, it may lead to non-responsiveness to anti-CLEVER-1 antibody therapy and other treatment options are likely to be better.

### CITED REFERENCES

[1] Chen D S, Mellman I. Elements of cancer immunity and the cancer-immune set point. Nature 2017; 541(7637): 321-30.
[2] Hegde P S, Karanikas V, Evers S. The Where, the When, and the How of Immune Monitoring for Cancer Immunotherapies in the Era of Checkpoint Inhibition. Clin Cancer Res 2016; 22(8):1865-74 doi 10.1158/1078-0432.CCR15-1507.
[3] Guerriero J L, Sotayo A, Ponichtera H E, Castrillon J A, Pourzia A L, Schad S, et al. Class IIa HDAC inhibition reduces breast tumours and metastases through anti-tumour macrophages. Nature 2017; 543(7645):428-32.
[4] Qian B Z, Pollard J W. Macrophage diversity enhances tumor progression and metastasis. Cell 2010; 141(1):39-51.
[5] Ries C H, Cannarile M A, Hoves S, Benz J, Wartha K, Runza V, et al. Targeting tumor-associated macrophages with anti-CSF-1R antibody reveals a strategy for cancer therapy. Cancer Cell 2014; 25(6):846-59.
[6] Quail D F, Bowman R L, Akkari L, Quick M L, Schuhmacher A J, Huse J T, et al. The tumor microenvironment underlies acquired resistance to CSF-1R inhibition in gliomas. Science 2016; 352(6288): aad3018.
[7] Kzhyshkowska J, Gratchev A, Goerdt S. Stabilin-1, a homeostatic scavenger receptor with multiple functions. J Cell Mol Med 2006; 10(3):635-49.
[8] Kzhyshkowska J, Workman G, Cardó-Vila M, Arap W, Pasqualini R, Gratchev A, et al. Novel function of alternatively activated macrophages: stabilin-1-mediated clearance of SPARC. J Immunol 2006; 176(10):5825-32.
[9] Karikoski M, Marttila-Ichihara F, Elima K, Rantakari P, Hollmen M, Kelkka T, et al. Clever-1/Stabilin-1 Controls Cancer Growth and Metastasis. Clinical Cancer Research 2014; 20(24):6452-64.
[10] Viitala M., Virtakoivu R., Tadauon S., Rannikko J., Jalkanen S., Hollmen M., Immunotherapeutic Blockade of Macrophage Clever-1 Reactivates the CD8+ T-cell Response against Immunosuppressive Tumors. Clin Canc Res. February 2019 doi: 10.1158/1078-0432
[11] Virtakoivu R, Rannikko J H, Viitala M, Vaura F, Takeda A, Lönnberg T, Koivunen J, Jaakkola P, Pasanen A, Shetty S, de Jonge M J A, Robbrecht D, Ma Y T, Skyttä T, Minchom A, Jalkanen S, Karvonen M K, Mandelin J, Bono P, Hollmen M. Systemic Blockade of Clever-1 Elicits Lymphocyte Activation Alongside Checkpoint Molecule Downregulation in Patients with Solid Tumors: Results from a Phase I/II Clinical Trial. Clin Cancer Res. 2021 Aug. 1; 27(15): 4205-4220.

1. A method for choosing a patient to anti-CLEVER-1 therapy prior to beginning anti-CLEVER-1 therapy, which anti-CLEVER-1 therapy comprises an administration of an agent capable of binding to CLEVER-1 in a patient, characterized in that the method comprises
providing a blood sample obtained from the patient at a first point in time prior to beginning anti-CLEVER-1 therapy,
measuring a level of one or more inflammatory marker selected from the group consisting of interferon gamma (IFNg), tumour necrosis factor alpha (TNF-α), interleukin 6 (IL-6), interleukin 8 (IL-8) and C-reactive protein (CRP) from the obtained sample, and
comparing the measured level of the inflammatory marker to a reference value of said inflammatory marker,
wherein the measured level of the said inflammatory marker, which is substantially within the reference values of the inflammatory marker or lower than the average reference value of the inflammatory marker, is an indication to choose the patient and begin the anti-CLEVER-1 therapy.

2. The method according to claim 1, wherein the patient is a cancer patient.

3. The method according to claim 1, wherein the anti-CLEVER-1 agent comprises anti-CLEVER-1 antibody.

4. The method according to claim 1, wherein the anti-CLEVER-1 agent comprises anti-CLEVER-1 antibody bexmarilimab (FP-1305) deposited under the accession number DSM ACC3361.

5. The method according to claim 1, wherein the method comprises measuring at least the level of interferon gamma (IFNg) and tumour necrosis factor alpha (TNF-α).

6. The method according to claim 1, wherein the method comprises measuring the level of interferon gamma (IFNg), tumour necrosis factor alpha (TNF-α), interleukin 6 (IL-6) and/or interleukin 8 (IL-8) in combination with measuring of the level of C-reactive protein (CRP).

7. The method according to claim 1, wherein the level of the inflammatory marker is measured from serum and/or plasma.

8. The method according to claim 1, wherein the anti-CLEVER-1 therapy is used for treating advanced solid tumours.

9. The method according to claim 3, wherein the anti-CLEVER-1 antibody is bexmarilimab.
